(19) United States
(12) Patent Application Publication
(10) Pub. No.: US 2021/0065841 A1
(43) Pub. Date: Mar. 4, 2021

(54) METHOD FOR DETERMINING ANDROGEN AND ANTI-ANDROGEN EFFECTS ON SUBSTANCES

(71) Applicant: Nanjing University, Nanjing City (CN)

(72) Inventors: Hongling LIU, Nanjing City (CN); Laihao SHI, Nanjing City (CN)

(21) Appl. No.: 17/043,693

(22) PCT Filed: Oct. 24, 2018
(86) PCT No.: PCT/CN2018/111676

§ 371 Date: Sep. 30, 2020

(30) Foreign Application Priority Data
Sep. 3, 2018 (CN) 201811017889.9

(51) Int. Cl. G16B 5/00; G16B 50/30; G16B 50/20; G16B 15/20

(52) U.S. Cl. CPC G16B 5/00; G16B 15/20; G16B 50/20; G16B 50/30

(57) ABSTRACT

The present invention provides a method of constructing a ligand-receptor protein complex to determine whether a substance has androgen or anti-androgen activity by utilizing the Surflex-Dock program of SYBYL to dock the ligand into the AR. Through the establishment of the binding model of dihydrotestosterone (DHT) and hydroxyflutamide (HFT) and AR, it is found the interaction is significantly weakened between other conformations to the H12 helix bound by other antagonists after simulation, which would reduce the stability of H12 helix, while the agonists can maintain the stability of H12. This method can be used to screen preliminarily different agonistic and antagonistic substances having a number of different structures.

### CROSS-REFERENCES TO RELATED APPLICATIONS

This application is a 371 application of the International Patent Application No. PCT/CN2018/111676 filed on Oct. 24, 2018, which claims priority from the Chinese Patent Application Number 201811017889.9 filed on Sep. 3, 2018, and the disclosure of which is incorporated herein by reference in its entirety.

### FIELD OF THE INVENTION

The present invention relates to the technical field of testing or analyzing by means of measuring chemical or physical properties, in particular to a method for determining androgen and anti-androgen effects on substances and an application thereof.

### BACKGROUND OF THE INVENTION

At present, the existing technology widely used in the industry is as follows: the endocrine disrupting effect of chemical substances is an important part of its comprehensive toxicity. With respect to the definition of the US EPA, endocrine disruptors are regarded as environmental substances that can affect the homeostasis, reproduction and development of organisms by interfering with the process of synthesis, secretion, transport, metabolism, binding and removal of endogenous hormones. Among them, environmental androgen is a kind of important pollutant. The substance which can mimic natural androgen so as to activate androgen receptor is viewed as androgen mimics, and the substances that inhibit natural androgen is viewed as anti-androgen. In recent years, it has been found in many wild animals, such as fish and birds, having symptoms with various degrees of reproductive disorders, malformations of sexual organs and male feminization. In the previous studies, the mechanism of this phenomenon have been focused on the environmental estrogen. Estrogens such as estrone, estradiol, equol and other estrogens discovered in field studies cannot explain the high incidence of hermaphrodite in wild barracudas in Liaodong Bay. However, p, p′-DDE and equol with anti-androgenic effects might be the reasons for this phenomenon in Liaodong Bay. The existing screening methods mainly include experimental methods and calculation methods, where the experimental methods can be further divided into in vitro methods and in vivo methods. In vitro experiments include cell proliferation experiments, reporter gene experiments, competitive binding experiments, and yeast two-hybrid experiments. In vivo experiments mainly utilize animal feeding, rodents (such as rats, etc.) hormone-dependent tissues (such as prostate, seminal vesicles, uterus, etc.) experiments for gain of weight after excision, and in vivo biomarker experiments, etc. There are advantages and disadvantages for both in vitro experiments and in vivo experiments. In vivo experiments have been developed and applied to various high-throughput testing and screening methods to deal with the huge number of potential hazardous compounds, as well as the huge cost and ethical issues from in vivo animal testing. However, because in vitro experiments cannot fully mimic the complicated mechanisms of distribution, absorption, metabolism, excretion process and other factors in the animals, the accuracy of the result obtained therefrom is not comparable to that obtained from in vivo tests. Further, both of these methods are time-consuming, labor intensive and cannot address the increasing amounts of chemicals. Therefore, there is an urgent need to develop a computational toxicology method for screening the chemicals with endocrine disrupting effects. The computational toxicology method refers to the development of mathematical or computer models by integrating data from different sources such as in vivo and in vitro experiments and computer simulations in order to better understand or predict the disrupting effects of compounds. The conventional quantitative structure-activity relationship (QSAR) has been widely used and achieved good results. However, in reality, although the structure of the compounds are similar, some of them are active while the rest are not. This makes conventional QSAR methods that rely solely on the structure to predict the effect unable to determine the presence or absence of activity. The molecular docking and the molecular dynamics are utilized to simulate the interaction between the ligand and the receptor bound to form a stable complex, which is able to identify the potential endocrine disruptors.

However, the existing molecular docking only focuses on the docking situation with a specific conformation. Conformational changes obtained by the molecular dynamics methods are performed within a limited time period, and only focus on a class of compounds with similar structures, and the pattern recognition is relatively simple. For example, Wang Xiaoxiang and others have applied molecular dynamics to screen nuclear receptor mediated endocrine disruptors, but this method only evaluates the fluctuation of the root mean square deviation after docking with the receptor and the prediction accuracy is low.

In summary, the problems in the prior arts are:

(1) The existing in vivo and in vitro screening methods are not able to address the increasing number of chemicals.
(2) The existing conventional QSAR cannot differentiate the activity; the molecular docking and the molecular dynamics methods often only focus on a specific conformation of the compound and conformation changes within a limited time period.

Challenges and objectives of solving the above technical problems:

Biological screening methods for the mimic/anti-androgen effects of each environmental organic pollutant are time- and cost-consuming. The existing virtual screening methods have the common problem in activity prediction or limited ability to predict activity of only one class of substances. An objective of the present invention is to provide a method for identifying the mimic/anti-androgenic activity of a various compounds with different structures basing on molecular dynamics analysis of the interaction and energy change between the ligand and the receptor and the allosteric effect of the receptor.

### SUMMARY OF THE INVENTION

With respect to the above problems in the existing technology, the present invention provides a method of constructing a ligand-receptor protein complex to determine whether a substance has androgen or anti-androgen activity, wherein the method for determining a substance having either androgen or anti-androgen activity involves a computer software, e.g., Surflex-Dock program of SYBYL, to dock a ligand into an androgen receptor (AR) of a subject, e.g., a human AR.

The method of constructing a ligand-receptor protein complex to determine whether a substance has androgen or anti-androgen activity further comprises following steps:

Step 1: Pre-treatment to the receptor protein before docking, which includes adding hydrogen atoms and assigning charges. The Automatic mode is applied to search for binding pockets when docking. The threshold value is 0.5, the bloat value is 0, and both are set for 17 as default values.

Step 2: 20 conformations are generated when each ligand is docked with the receptor protein. The structure with highest score is regarded as the most likely biologically active conformation, and this conformation is used as the initial conformation for MD simulation.

The method of constructing a ligand-receptor protein complex to determine whether a substance has androgen or anti-androgen activity further comprises following steps:

Step 1: The Sketch Module in SYBYL7.3 is used to construct the structures of the ligand molecule and the positive control and to minimize the energy of the ligand molecule. The Powell method is utilized to optimize the energy, followed by giving Gasteiger-Hückel charge, using standard Tripos molecular force field, and performing energy optimization. The standard restrained energy used for optimizing energy in standard Tripos molecular force field is 0.001 kcal/(mol Å), and the maximum number of iterations are 1000 times.

Step 2: The androgen receptor sequence is imported into the Swiss-Model platform, and the activated androgen receptor conformation is used as the template for the homology modeling to establish an activated conformation of human AR.

Step 3: Surflex-Dock module in SYBYL is used to dock the ligand molecules to the AR. The receptor protein is pre-treated before docking including adding hydrogen atoms and assigning charges, and each binding between the ligand and the receptor generates 20 conformations. The highest scoring conformation is the most likely biologically active conformation for the intial conformation in molecular dynamics (MD) simulation.

Step 4: The CHARMM27 force field is used in MD simulation, followed by filling in TIP3P water molecule layer around ligand-receptor protein complex system. The distance between the ligand-receptor protein complex system and the margin of the solvent is 1.5 nm, and chloride ion is added to neutralize the charge in the system. The steepest descent method is used to optimize energy in the system. The system is simulated in 40 ps, heating gradually from 0K to 300K, and equilibrating under lns at 300K at 1 atm. Then, the MD simulation is carried out under 30 ns with the time step 2 fs and recorded the trajectory every 2 ps.

In summary, the advantages and positive effects of the present invention are:

Through the establishment of a binding model of dihydrotestosterone (DHT) and hydroxyflutamide (HFT) and AR, it is found the interaction is significantly weakened between other conformations to the H12 helix bound by antagonists after simulation, which would reduce the stability of H12 helix, leading to a small increase in the distance between the H12 helix and the ligand binding domain, while the agonist can maintain the stability of H12. This method can be used to screen different agonist and antagonistic substances having a number of different structures.

The present invention provides a method of constructing a ligand-receptor protein complex to determine whether a substance with androgen and anti-androgen effects via a molecular dynamics model, simulating the interaction and the change in distance between the H12 chain of the androgen receptor and the ligand binding domain so as to identify the mimic/anti-androgen receptor properties of the organic substances. Molecular dynamics simulation is a research method which includes simulating the movement process of a molecular system, calculating the structure and property of said system, and illustrating the trajectory movement of a compound in space so as to simulate the microscopic behavior of molecules. This method can also be used for re-docking the molecular docking system and exploring the actual conformation of the receptor, avoding the problem during the experiments.

### BRIEF DESCRIPTION OF THE DRAWINGS

FIG. 1 shows the flow chart to the method for determining substances with androgen and anti-androgen effects in one embodiment of the present invention.

FIG. 2 shows the flow chart to the method for determining substances with androgen and anti-androgen effects in another embodiment of the present invention.

FIG. 3 shows the stability evaluating diagram for distinguishing the antagonists and agoinists for the androgen receptor in one embodiment of the present invention.

FIG. 4 shows diagrams of the residue patterns of AR interacting with the ligands, DHT, HFT, TBB, TBCO, TBPH, TBBPA, and BDE155, for distinguishing the antagonists and agoinists for the androgen receptor in one embodiment of the present invention.

FIG. 5 is a schematic diagram of the distance between H874, W741, and R871 to the H12 helix of the receptor bound with DHT before simulation for distinguishing the antagonists and agoinists for the androgen receptor in one embodiment of the present invention.

FIG. 6 is a schematic diagram of the distance between H874, W741, and R871 to the H12 helix of the receptor bound with DHT after simulation for distinguishing the antagonists and agoinists for the androgen receptor in one embodiment of the present invention.

FIG. 7 is a schematic diagram of the distance between R871 to the H12 helix of the receptor bound with HFT for distinguishing the antagonists and agoinists for the androgen receptor in one embodiment of the present invention.

FIG. 8 is a schematic diagram of the binding energy for distinguishing the antagonists and agoinists for the androgen receptor in one embodiment of the present invention.

### DETAILED DESCRIPTION

In order to make the objectives, technical solutions, and advantages of the present invention clearer, the present invention will describe below in further embodiments. It should be understood that the specific embodiment described hereinafter are only used to explain the present invention, but not to limit the present invention.

Environmental androgen-like substances and anti-androgen substances are widespread in the environment. Although there are trace amounts of said substances in the environment, they can seriously interfere with the endocrine function of organisms, resulting in androgyny. In the face of increasing potential androgen receptor interference substances, it is urgent to develop a rapid screening method. The present invention provides a method for distinguishing substances with androgen and anti-androgen effects by simulating the binding process of the androgen receptor and a series of ligands via molecular dynamics.

The principle of the present invention is described in detail along with drawings.

In accordance to one embodiment of the present invention, a method of constructing a ligand-receptor protein complex to determine whether a substances with androgen and anti-androgen effects is provided by utilizing the Surflex-Dock program of SYBYL to dock the ligand into the AR.

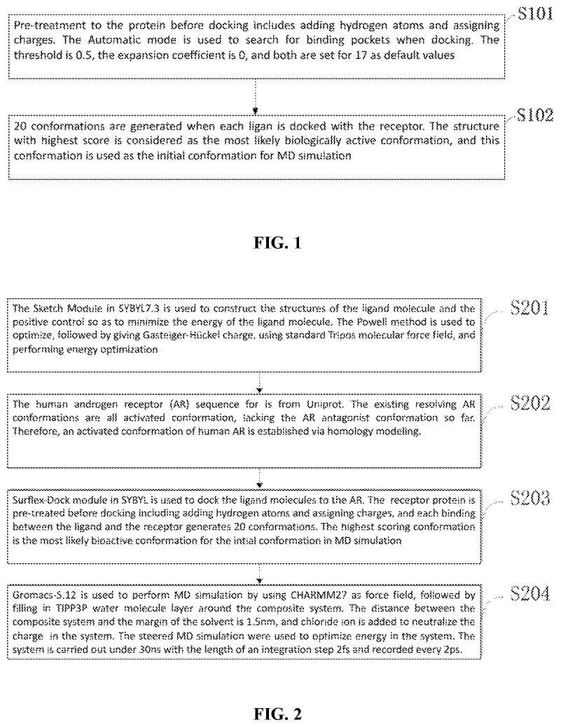

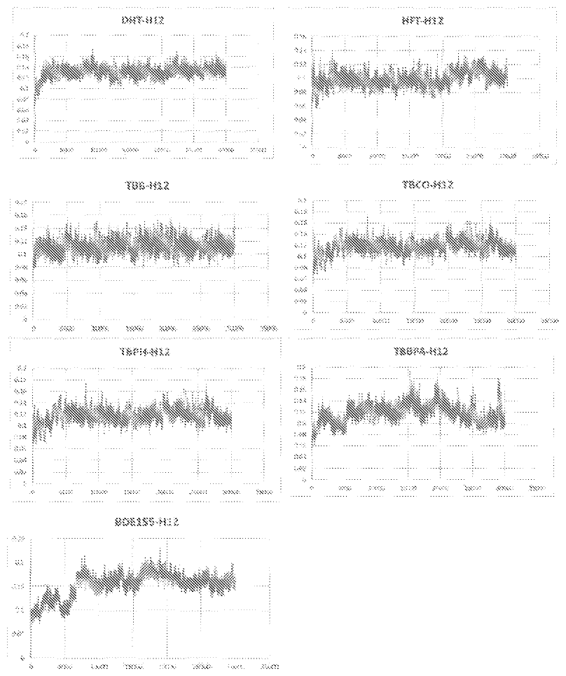

Refering to FIG. 1, the method for constructing a ligand-receptor protein complex to determine whether a substance with androgen and anti-androgen effects in one embodiment of the present invention further includes following steps:

S101: Pre-treatment to the receptor protein before docking includes adding hydrogen atoms and assigning charges. The Automatic mode is applied to search for binding pockets when docking. The threshold value is 0.5, the bloat value is 0, and both are set for 17 as default values;

S102: 20 conformations are generated when each ligand is docked with the receptor protein. The structure with highest score is considered as the most likely biologically active conformation, and this conformation is used as the initial conformation for MD simulation.

Refering to FIG. 2, the method of constructing a ligand-receptor protein complex to determine whether a substance with androgen and anti-androgen effects in another embodiment of the present invention further includes following steps:

S201: The Sketch Module in SYBYL7.3 is used to construct the structures of the ligand molecule and the positive control to minimize the energy of the ligand molecule. The Powell method is utilized to optimize, followed by giving Gasteiger-Hückel charge, using standard Tripos molecular force field, and performing energy optimization;

S202: The human androgen receptor (AR) sequence is from Uniprot. The existing resolving AR conformations are all activated conformation, lacking the antagonist conformation of AR so far. Therefore, an activated conformation of human AR is established via homology modeling.

S203: Surflex-Dock module in SYBYL is used to dock the ligand molecules to the AR. The receptor protein is pre-treated before docking including adding hydrogen atoms and assigning charges, and each binding between the ligand and the receptor generates 20 conformations. The highest scoring conformation is the most likely biologically active conformation for the intial conformation in MD simulation;

S204: Gromacs-5.12 is applied to perform MD simulation by using CHARMM27 as force field, followed by filling in TIP3P water molecule layer around the ligand-receptor protein complex system. The distance between the ligand-receptor protein system and the margin of the solvent is 1.5 nm, and chloride ion is added to neutralize the charge in the system. The steepest descent method were used to optimize energy in the system. The system is carried out under 30 ns with the time step 2 fs and recorded every 2 ps.

The ligand molecules in the present invention includes, for examples, but not limited to TBB, TBCO, TBPH, TBBPA, BDE155, wherein TBB, TBCO, TBPH and TBBPA have androgen receptor antagonistic properites in the previous reports.

In one embodiment, the standard restrained energy used for optimizing energy in standard Tripos molecular force field is 0.001 kcal/(mol A), and the maximum number of iterations are 1000 times.

In another embodiment, an activated conformation of human AR is also provided, wherein the structure of activated conformation of human AR is constructed by using the template of AR-LBD binding with 5α-DHT from the Swiss-model platform.

In one embodiment, it is also provided that the Automatic mode is used to search for binding pockets when docking, wherein the threshold value is 0.5, and the bloat value is 0, and both of them are default values.

In one embodiment, the steepest descent method were used to optimize energy in the system, wherein the system is simulated in 40 ps, heating gradually from 0K to 300K, and equilibrating under lns at 300K at 1 atm.

1. Energy Caculation for Molecular Mechanics Poisson-Boltzmann Surface Area (MMPBSA):

In order to quantify the interaction between the ligand and the receptor, the binding energy ligand and the receptor was calculated. The free energy is calculated by MMPBSA method and the trajectory file gerenrated by the binding between the ligand and the receptor. The formula for calculating the free energy by MMPBSA is provided below:

ΔG_{binding}=G_{complex}−(Gₚᵣₒₜₑᵢₙ+G_{ligand});

In the above formula, G_{complex} represents the total free energy of the protein-ligand complex, and Gₚᵣₒₜₑᵢₙ and G_{ligand} represent the total energy of the separated protein and ligand in the solvent, respectively. Further, each Gₓ can be calculated by the following formula:

Gₓ=E_{MM}−TS+Gₛₒₗᵥₐₜᵢₒₙ;

In the above formula, E_{MM} is the average molecular mechanical energy; TS is the contribution from the entropy, wherein T and S represents temperature and entropy, respectively, and Gₛₒₗᵥₐₜᵢₒₙ is the solvation free energy. E_{MM} includes bond energy, electrostatic interaction, and van der Waals interaction, showing in the following formula:

E_{MM}=E_{bond}+E_{electrostatic}−E_{vdW};

The solvation free energy includes two parts: polar and non-polar solvation free energy, showing in the following formula:

Gₛₒₗᵥₐₜᵢₒₙ=Gₚₒₗₐᵣ+Gₙₒₙ₋ₚₒₗₐᵣ;

wherein Gₙₒₙ₋ₚₒₗₐᵣ is calculated by solvent accessible surface area (SASA) model.

2. Results

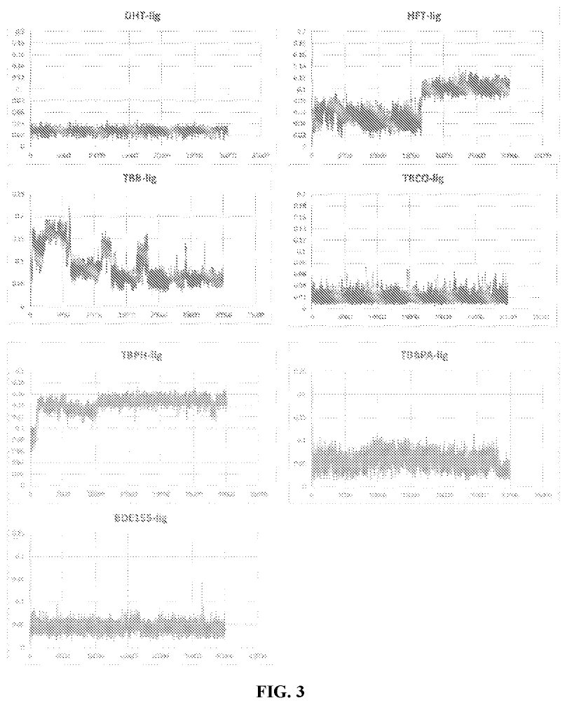

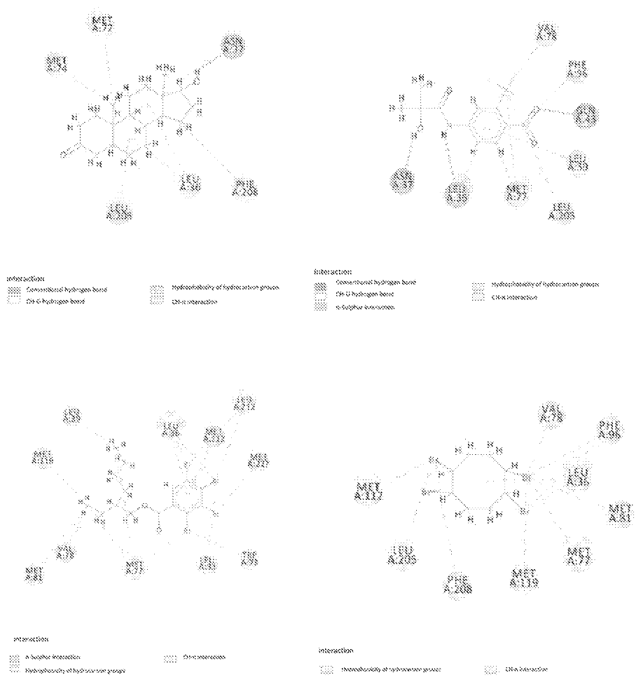

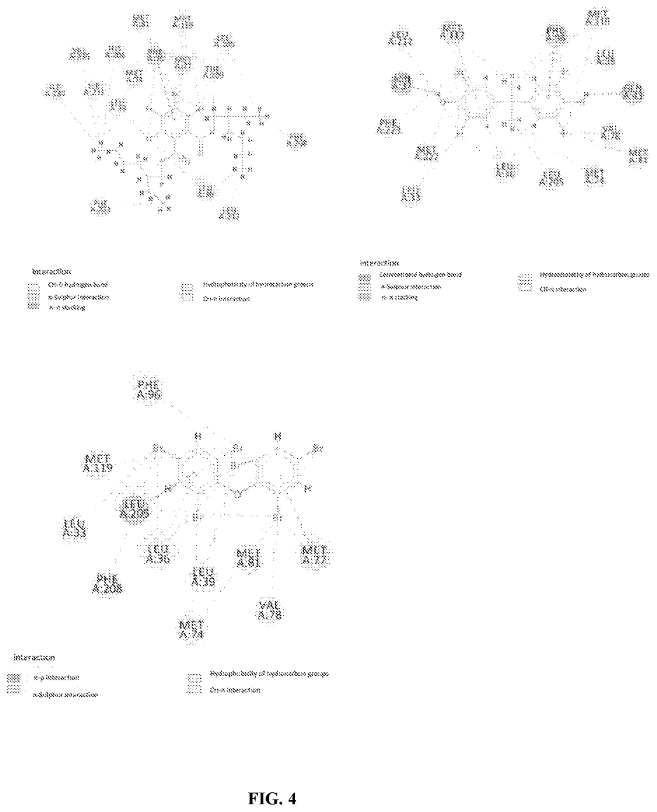

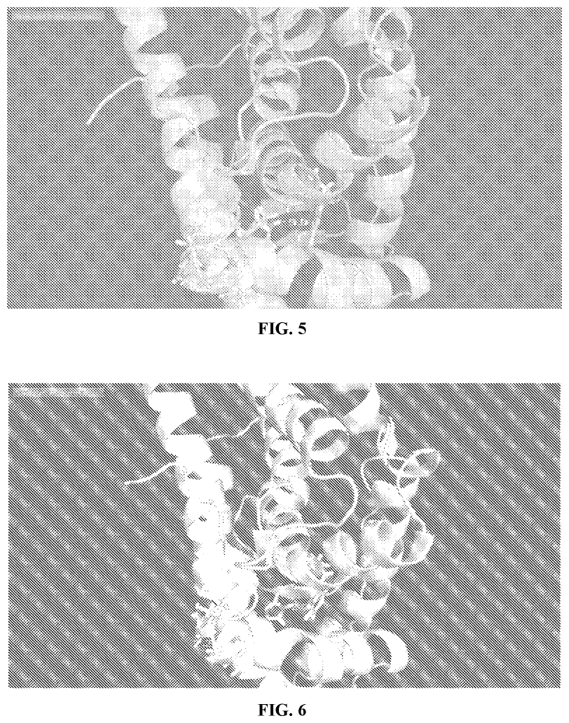

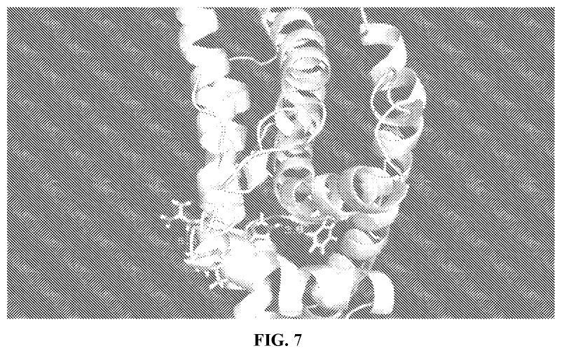

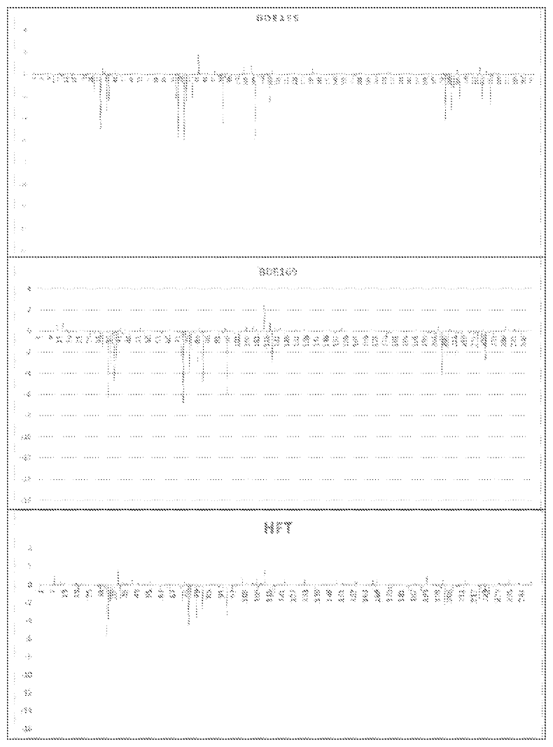

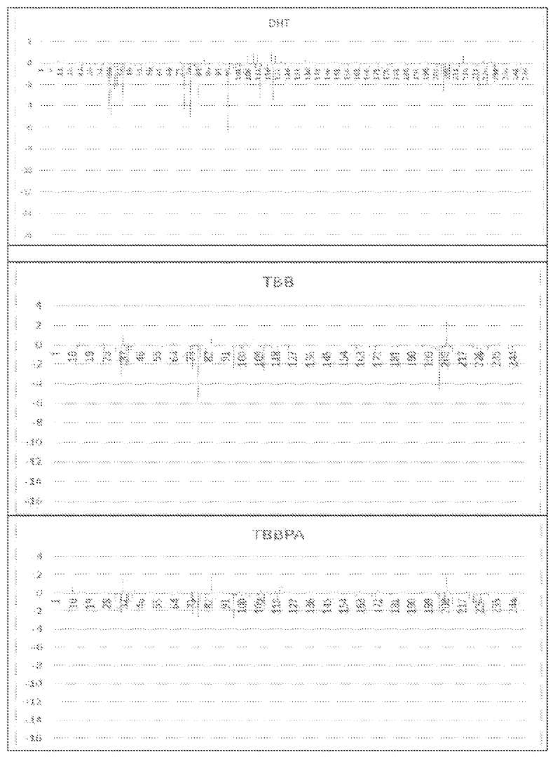

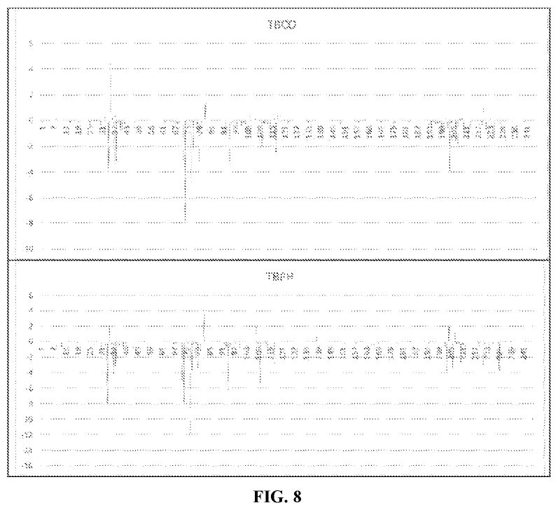

Refering to FIG. 3 to FIG. 8, after the simulation, conformations before and after the simulation were extracted for superimposition comparison, and it was found that the conformations of each receptor did not change significantly after 30 ns simulation. Then, the average root mean squared differences (RMSD) of H12 chain and ligand molecules in all system are analysed. And all system equilibrated before 30 ns. After the equilibration, the change of H12 chain is approximately 0.1 mm, and the change of RMSD of BDE-155 is close to 0.15, which is significantly higher than the other ligands. However, this cannot be a good distinction. It can be shown from the RMSD of the ligand, except for TBCO, the RMSD of the antagonist ligand cannot reach equilibrium before 15 ns or the RMSD is higher than 0.1 nm after equilibrium. The agonist can reach equilibrium quickly after binding, and the RMSD is less than 0.1 nm after equilibrium.

Ligand-Receptor Interaction Residues

According to the residues of AR interacting with ligands, the residues that interact with these substances are less conservative. It is not able to distinguish antagonists from agonists by identifying the form of the residues interacting with the ligand.

Hydrogen Bond in H12 Chain

Drug resistance to anti-androgen drugs such as flutamide and enzalutamide is due to the mutations in the residue sequence of the AR receptor. Further, the mutations of L701H, W741L, H874Y, T877A and M895T on AR would cause the changes of the hydrogen bond network between these amino acid residues so as to make anti-androgen drugs such as flutamide to bear drug resistance. Therefore, the interaction changes between these amino acid residues are investigated after binding these ligands to the androgen receptor.

Before and after the simulation, the conformational changes were not significant. In the intial state, the C-terminus of the receptor H12 chain forms two hydrogen bonds with the H11 chain. After binding with DHT and other agonists, the hydrophilic groups of W741 are toward H874, reducing the distance from 3.9 A to 2.2 A, which would lead the residue R203 of H874 to shorten the distance with H12 helix to approximately 2.2 A so as to maintain the hydrogen bonds of H11 helix and H12 helix. Comparing with the initial conformation, M895 on H12 helix is approaching to the LBD region.

After simulating the receptor binding with antagonist HFT for 30 ns, the hydrophobic benzene ring of W741 is toward H874 or W741 is away from H874, i.e. make H874 away from W741, and the residue R871 of H874 is away from H12 chain, and increasing the distance to 5.8 A. The hydrogen bonds between H11 helix and H12 helix were disappear, which indicates a more unstable structure of H12 due to the binding of the antagonist. Meanwhile, comparing with the intial conformation, M895 on H12 helix is away to the LBD region.

In view of the binding energy, the binding energy of amino acids on H12 helix bound with antagonists is significantly lower than that bound with agonists, which indicates the decrease of the firmness interaction between H12 and LBD region bound with antagonist and H12 tends to be unstable comparing with the AR receptor bound with agonists.

The above descriptions are only preferred embodiments of the present invention and are not intended to limit the present invention. Any modification, equivalent replacement and improvement made within the spirit and principle of the present invention shall be included in the protection of the present invention.

1. A method of constructing a ligand-receptor protein complex to determine whether a substance has androgen or anti-androgen activity, the method comprising:
providing a ligand and a modified androgen receptor protein;
docking the ligand to the androgen receptor protein comprising performing Surflex-Dock program of SYBYL;
modifying the androgen receptor protein comprising adding hydrogen atoms and assigning charges, searching for a binding pocket for said ligand with a threshold value of 0.5 and a bloat value of 0 prior to said docking;
generating 20 different conformations of the modified androgen receptor protein when the ligand docks to the receptors thereof;
selecting the highest score conformation according to the scores obtained among the 20 different conformations,
wherein the highest score conformation is the most likely biologically active conformation of the androgen receptor protein for a subsequent screening of potential candidates of the substance having the androgen activity.

2. (canceled)

3. The method of claim 1, further comprising:
constructing a structure of the ligand by Sketch Molecule module in sybyl7.3 and optimizing the structure including using Powell method to optimize, giving Gasteiger-Hückel charge, using standard Tripos molecular force field, and performing energy optimization;
performing a homology modelling to obtain an activated conformation of androgen receptor;
performing a pre-treatment to the androgen receptor protein comprising adding hydrogen atoms and assigning charges; docking the ligand to the androgen receptor protein by performing Surflex-Dock program of SYBYL; generating 20 conformations when the ligand dockes to the receptors; scoring the conformations to obtain a highest score conformation, wherein the highest score conformation is identified as a potential biological conformation, and the conformation is used as an initial protein receptor-ligand complex conformation for molecular dynamic simulation;
applying CHARMM27 force field to a molecular dynamic simulation comprising filling TIP3P water molecule in at least 1.5 nm away from the surface of the protein receptor-ligand complex, adding at least one chloride ion to neutralize the charge in the complex, performing steepest descent method to the complex to optimize the energy under 30 ns with a time step 2 fs and recording a trajectory every 2 ps.

4. The method of claim 3, wherein the standard restrained energy of said using standard Tripos molecular force field and performing energy optimization is 0.001 kcal/(mol Å), and the maximum number of iterations are 1000 times.

5. The method of claim 3, wherein said performing energy optimization is simulating in 40 ps, heating gradually from 0K to 300K, and equilibrating under 1 ns at 300K at 1 atm.

6. The method of claim 3 further comprising:
obtaining a binding energy by performing MMPB SA calculation;
analyzing a change in distance between a binding region of the ligand and amino aicds on a H12 helix when the protein receptor-ligand complex interact with the substance;
wherein decrease in the distance indicates the substance having androgen activity, whereas increase in the distance indicates the substance having anti-androgen activity so as to determine whether said substance is androgen or anti-androgen-like compound or molecule.

7. The method of claim 6, wherein the decrease in the distance is approximately from 3.9 A to 2.2 A.

8. The method of claim 6, wherein the increase in the distance is approximately from 3.9 A to 5.8 A.
